(19) United States
(12) Patent Application Publication
(10) Pub. No.: US 2011/0027195 A1
(43) Pub. Date: Feb. 3, 2011

(54) COMPOSITION FOR DENTAL BARRIER COMPRISING AT LEAST ONE MONOMER, AT LEAST ONE POLYMERIZATION INITIATING SYSTEM, AND AT LEAST ONE INDICATOR ENABLING THE POLYMERIZATION REACTION TO BE MONITORED

(75) Inventors: Vincent MAURAT, PESSAC (FR); Marguerite FOURNIE, ANDERNOS-LES-BAINS (FR)

Correspondence Address: LUCAS & MERCANTI, LLP, 475 PARK AVENUE SOUTH, 15TH FLOOR, NEW YORK, NY, 10016, US

(73) Assignee: PRODUITS DENTAIRES PIERRE ROLLAND, Merignac (FR)

(21) Appl. No.: 12/581,529

(22) Filed: Oct. 19, 2009

(30) Foreign Application Priority Data
Jul. 30, 2009 (FR) 0955372

(51) Int. Cl. A61K 8/44; B05D 3/06; A61K 8/365; A61K 8/36; A61Q 11/02

(52) U.S. Cl. 424/54; 427/2.29; 424/55; 424/49

(57) ABSTRACT

The present invention relates to a composition for a dental barrier, comprising at least one monomer of methacrylate or methacrylic derivative type, at least one thickener, a photochemical means of initiating polymerization, and at least one colored indicator.

The invention also relates to a method for making a dental barrier, the dental barrier thus obtained, and the use of said dental barrier for protecting the dental tissues, in particular the gums, during treatment in the dental office, in particular an office-bleaching treatment.

The present invention relates to a novel dental barrier composition comprising at least one monomer, at least one polymerization initiating system, and at least one indicator enabling the polymerization reaction to be monitored, as well as the use of this colored indicator for monitoring the polymerization reaction during the production of dental barriers.

The compositions according to the invention are intended to be used by qualified dental practitioners on their patients, during any operation requiring protection of an area of the mouth.

### PRIOR ART

Dental barriers, also called dental dams, have been in use in various forms for many years.

U.S. Pat. No. 2,092,549 describes a premolded dental dam made of latex, which is placed on the area to be protected. These dams have the drawback that they do not adhere to the area to be protected, and so do not remain properly in place during the dental procedures that are to be carried out.

U.S. Pat. No. 7,157,502 in the name of Pulpdent, describes a method of forming a polymerizable dental barrier, consisting of polymerizing a polymer of high molecular weight on the patient's dental tissue. It is noted that in this document the compositions comprise polymers and not monomers as starting product, in order to minimize the heat released during polymerization, so that it is less painful for the patient.

Patent EP 0 991 368 in the name of Ultradent discloses photopolymerizable compositions for making a dental barrier, comprising at least one monomer, at least one hardener, and at least one organic agent for reducing the strength of adherence and/or an agent for intensifying the adherence to the tissue and/or a reflecting material. This composition therefore does not allow the polymerization of the dental barrier which is being made to be monitored, which is rather inconvenient during practical application of the composition.

However, the applicant has looked for a new composition for making dental barriers, preferably by photochemistry, permitting the problems encountered with the compositions of the prior art to be solved.

In fact, owing to the heat generated during polymerization of a photopolymerizable dental dam, dentists are often in the habit of limiting patients' discomfort by polymerizing the material for intervals of one or two seconds. Now, these materials must be polymerized for 10 to 20 seconds using intense light of the appropriate wavelength. There is therefore a risk of leaving certain areas unpolymerized and therefore a risk of the patient ingesting toxic methacrylate derivatives that are not trapped by the polymerization reaction, or in certain uses, for example relating to the area of whitening, of burning or even necrotizing the unpolymerized areas. Moreover, it is necessary for the treated area to be isolated correctly by perfect polymerization, as the products applied subsequently can generate chemical or thermal aggression on the areas that are not correctly protected. More generally, the polymerizable protection systems are also used for isolating a treatment area during dental treatment.

### AIMS OF THE INVENTION

Thus, the main aim of the present invention is to provide a novel photopolymerizable composition, for making a dental barrier, making it possible to isolate the dental tissues, where the state of polymerization can be monitored visually by the user, enabling the user to ensure that polymerization is complete on all of the dental barrier formed, and thus achieve optimal protection of all the tissues that are to be protected.

Another main aim of the present invention is to provide a photopolymerizable composition that comprises monomers as the base compounds, rather than compounds that are already polymerized, making it possible to obtain a method of manufacture of the composition that is more simple and less expensive, and can be used on an industrial scale.

The present invention finally also aims to provide a photopolymerizable composition which, on the basis of its composition and monitoring of its polymerization, makes it possible to avoid any sensation of burning or of discomfort inherent in the polymerization reaction, which produces heat in situ.

### DESCRIPTION OF THE INVENTION

A first object of the present invention relates to a novel precursor composition for dental barriers, comprising at least one monomer of the methacrylate or methacrylic derivative type, at least one thickener, a photochemical means of initiating polymerization, and at least one colored indicator for monitoring the polymerization reaction.

This composition thus constitutes a precursor enabling a dental barrier to be obtained after photopolymerization of said composition.

According to a preferred embodiment, the composition according to the invention comprises:
50 to 90 wt. % of at least one monomer of the methacrylate or methacrylic derivative type,
10 to 49 wt. % of at least one thickener,
up to 2 wt. % of a photochemical means of initiating polymerization, and
up to 5 wt. % of a colored indicator for monitoring the polymerization reaction.
   The percentages are indicated by total weight of the composition.

In the composition according to the invention, on the one hand, said at least one monomer can be a methacrylic monomer.

Preferably, said monomer can be selected from the alkylmethacrylate, alkylhydroxymethacrylate, and alkylaminomethacrylate derivatives.

Particularly preferably, said monomer can be selected from diurethane dimethacrylate, triethylene glycol dimethacrylate, bisphenol A dimethacrylate, methyl methacrylate, and mixtures thereof, in particular the mixture consisting of diurethane dimethacrylate and triethylene glycol dimethacrylate.

In the composition according to the invention, on the other hand, said at least one thickener can be selected from cetyl alcohol, anhydrous colloidal silica, glycerol dibehenate, alginic acid, sodium alginate, potassium alginate, ammonium alginate, calcium alginate, propane-1,2-diol alginate, agar, carrageenan, furcellaran, carob gum, oat gum, guar gum, tragacanth, gum arabic, xanthan gum, gum karaya, gellan gum, sorbitol, mannitol, glycerol, polyoxyethylene stearate, polyoxyethylene-20-sorbitan monolaurate, polyoxyethylene-20-sorbitan monooleate, polyoxyethylene-20-sorbitan monopalmitate, polyoxyethylene-20-sorbitan monostearate, polyoxyethylene-20-sorbitan tristearate, pectin, gelatin, cellulose, methyl cellulose, ethyl cellulose, hydroxypropyl cellulose, hydroxypropylmethyl cellulose, methylethyl cellulose, carboxymethyl cellulose, salts of fatty acid, fatty acid mono- and diglycerides, esters of mono- and diglycerides, sugars of esters of fatty acids, sugar glycerides, polyglycerol esters of fatty acids, polyglycerol polyricinoleate, propylene glycol esters of fatty acids, mixtures of glycerol and propylene glycol esters of lactic acid and of fatty acids, esterified soya oil, sorbitan stearate, sorbitan tristearate, sorbitan monolaurate, sorbitan monooleate, sorbitan monopalmitate and mixtures thereof.

Preferably, said thickener can be selected from cetyl alcohol, anhydrous colloidal silica, glycerol dibehenate, and mixtures thereof.

In particular, according to the invention, said thickener can be selected from the commercial products constituted essentially of one or more of the products mentioned above. For example Aerosil 200®, which is essentially constituted of anhydrous colloidal silica, can be used as anhydrous colloidal silica.

According to the invention, the photosensitive adhesive composition includes a photochemical initiator whose composition can vary. Multiple initiators can be used, and it is then called a multicomponent initiating system, which can give rise to a hybrid or dual polymerization or chain crosslinking.

The examples given do not limit the scope of the invention in any way and should assist the formulator in choosing from the compounds that are available, notably in relation to the desired use of the composition according to the invention.

The initiators can be constituted of at least one photoinitiator that is able to initiate a mechanism of polymerization under the action of crosslinking radiation of wavelength λ. In the present text λ denotes both radiation of a single wavelength and a wavelength range centered on the stated value. Moreover, the expression “crosslinking radiation” denotes electromagnetic radiation that is able to stimulate the generation of free radicals.

According to a first aspect of the initiators, they are photoinitiators.

According to a first variant of this aspect, the photoinitiator is of the type that is able to generate free radicals via a mechanism of homolytic photocleavage. The photoinitiators that give rise to such processes belong to various families and the photoinitiators according to the invention can be selected from benzyl dialkyl ketal, benzoin ether, α-hydroxy, α-alkyl phenyl ketone, benzoyl cyclohexanol, trimethylbenzoyl phosphine oxides and more generally bisacylphosphine oxides, α-amino thioalkylphenyl ketone, α-amino morpholino-phenyl ketone, sulfonic esters of α-hydroxy methylbenzoin, and their derivatives. There are also many other photocleavable initiators in which radicals are generated after a series of consecutive processes of homolytic cleavage. The main examples known by a person skilled in the art are benzoyl oxime esters, arylsulfides, peroxides, peroxides containing a chromophore such as benzophenone or any alkylphenone, disulfides, ketosulfides and azo compounds such as AIBN (azobisisobutyronitrile) and azobenzoins.

According to a second variant of this aspect, the photoinitiator is of the type that is able to create free radicals by an atom-stripping mechanism. The commonest class of these photoinitiators is that in which a proton is stripped from a substrate in the course of photoreduction of an nn* triplet state of the photoinitiator, the main examples being the derivatives of benzophenone, of thioxanthones, of benzyl, of 1,2-diketones, such as camphorquinone, and of ketocoumarins. The formulator has other photosensitive compounds at his disposal, the onium salts such as notably the triarylsulfonium salts, alkylarylsulfonium salts and diarylhalonium salts. These compounds are generally much used as photoinitiator of cationic polymerization; however, the interaction of the triplet state with a proton donor produces free radicals that can also initiate a radical process.

According to a third variant of this aspect, the photoinitiator is of the photoreducible type. Free radicals are created following electron transfer. The families of compounds functioning in this way are chromophores such as diaryl ketone, camphorquinone, ketocoumarin or aromatic dyes such as xanthene, fluorone, thioxanthone, thiazine, acridine, anthraquinone, cyanine, merocyanine, benzopyran. The family of photoreducible aromatic dyes is quite particularly likely to be of interest to the formulator of dental resins, as these photoreducible aromatic compounds are excitable at visible wavelengths.

According to a second aspect of the initiators, the initiating system can be a two-component system (photoinitiator+coinitiator). Within the scope of the present invention, description of this aspect is limited to the case of radical chain polymerization, in which the two components between them generate electron transfer under irradiation at wavelength λ. This is just a special case of the concept of two-component photoinitiating systems.

One possibility is the combination of a photosensitive electron acceptor species with an electron donor species. This combination has generally been described most often, and numerous combinations can be envisaged. Thus, the photosensitive electron acceptor species include all of the families previously disclosed with regard to photoreducible photoinitiators. In this strategy, the electron donor species accelerates radical polymerization in a two-stage process, namely electron transfer followed by proton transfer. There are a great many species that are able to interact with the photoreducible species and we may mention in particular the compounds comprising an activated nitrogen atom, such as triethanolamine, a tertiary amine or a tertiary arylamine, in particular N,N-dimethyl-p-toluidine, N,N-diethanol-p-toluidine, N,N-dimethyl-sym(m)xylidine, 3,5-di-tert-butylaniline or N,N-dimethyl-p-ethyl aminobenzoate, compounds comprising an activated nitrogen atom and an activated sulfur atom such as thiazole derivatives, in particular mercaptobenzothiazole, boron salts (borates) such as derivatives of tetraphenyl borate and, more preferably, derivatives of butyltriphenyl borate. Owing to strong absorption below a wavelength of 300 nm and “tailing” of the spectrum in the visible range beyond a wavelength of 400 nm, or even 430 nm in certain cases, these compounds are photosensitive both in the UV range and in the visible range.

According to a third aspect of the initiators, the initiating system is a photoinitiating system comprising, in addition to the photoinitiator proper, at least one species, designated photosensitizer, that is able to photosensitize the initiator or initiators by creating active sites (free radicals). In this case, the interaction between the photosensitizer in one of its excited states with the photoinitiator can, principally and without limiting the description, be of two kinds. The photosensitization operates either by singlet state-singlet state or triplet state-triplet state energy transfer, or by photoinduced electron transfer with the photosensitizer, or by both at the same time.

If at least one of the photoinitiators is of the type that is able to generate free radicals by a mechanism of homolytic photocleavage, there are then a certain number of systems described in the literature that can accelerate this process. Thus, an important class of these initiators comprises the peroxides, but the mechanisms of photosensitization are very varied. For example, benzoyl peroxide and decanoyl peroxide can be photosensitized by energy transfer from the triplet state of compounds such as anthracene, acetophenone, methoxy- and cyanobenzophenones, combined with formation of a charge transfer complex. In other cases, it is a question of electron transfer, for example between a thioxanthene and 3,3′,4,4′-tetra-(t-butylperoxycarbonyl)-1-benzophenone. These examples are only given as a guide, bearing in mind the variability of the mechanisms of sensitization depending on the operating conditions and the chemical species involved.

Other important examples relate to the interaction that occurs between an α-amino acetophenone such as α-morpholino thiomethylphenyl ketone, and a photosensitizer of the thioxanthone type. A particular feature of this system is that it can function both by energy transfer from the triplet state of the thioxanthone and by electron transfer.

It is also possible to photosensitize certain two-component systems that combine an electron donor and an electron acceptor. In particular, the triple system of onium salt, “intermediate” photoreducible sensitizer and electron donor, is very effective for initiating acrylic formulations. Notably we can use the following combinations between:

An onium salt selected from the diaryliodonium salts and the triarylsulfonium salts,

A photosensitive and photoreducible intermediate species such as diaminoaryl ketone, ketocoumarin, thioxanthone, xanthene, fluorone, thiazine, acridine, anthraquinone, cyanine, merocyanine and benzopyran and their derivatives, and

An electron donor such as the compounds comprising an activated nitrogen atom, for example a tertiary amine and a tertiary arylamine, in particular N,N-dimethyl-p-toluidine, N,N-diethanol p-toluidine, N,N-dimethyl-sym(m)xylidine, 3,5-di-tert-butylaniline or N,N-dimethyl p-ethyl aminobenzoate, the compounds comprising an activated nitrogen atom and an activated sulfur atom such as thiazole derivatives, in particular mercaptobenzothiazole, boron salts (borates), for example tetraphenyl borates or butyltriphenyl borates, and other salts of the tetraalkylammonium tetraorganyl borates type.

The following system that is regarded as the “intermediate” species or the onium salt as the photosensitizer, can be considered differently.

Moreover, another series of examples applicable within the scope of the invention relates to the photosensitization of an onium salt, of the triarylsulfonium salt or diarylhalogenium salt type, used without combination, with an electron donor of the tertiary amine or borate salt type, optionally combined with an appropriate proton donor.

The formulator can then optionally select the photosensitizer by addition of a photosensitive compound permitting triplet state-triplet state energy transfer, selected from acetone, 1-indone, acetophenone, 3-trifluoromethyl-acetophenone and xanthone, or by incorporating a photosensitive compound permitting electron transfer with an onium salt selected from anthracene, pyrene, perylene, aromatic ketones such as benzophenone, Michler ketones, xanthones, thioxanthones, derivatives of dimethylaminobenzylidine, phenanthraquinones, eosin, ketocoumarins, acridines and benzofurans.

According to a last aspect of the initiators of the polymerization reaction, they are initiators of the photochemical type, whose composition can include at least one photoinitiator, at least one coinitiator, at least one photosensitizer or, more generally, any suitable combination of the various potential components of a photoinitiating system, as described previously, with the aim of increasing its efficacy and/or of modifying its range of absorption of electromagnetic radiation.

In the composition according to the invention, said photochemical means of initiating polymerization is preferably selected from camphorquinone, ethyl-4-dimethylaminobenzoate, bisacylphosphine oxide, 2,4,6-trimethylbenzoyldiphenylphosphine oxide, and the camphorquinone/ethyl-4-dimethylaminobenzoate system.

According to the invention, the system for monitoring polymerization is a visual system based on color change of a colored indicator contained in the composition. This indicator is able to indicate a change of the medium, for example a change in pH, the redox reaction or a phase change. In our case, the polymerization reaction induces a change of medium, i.e. a change of potential in the case of an indicator of redox reaction or of pH in the case of a pH indicator, which is observed by inspection of the colored indicator.

The compositions according to the invention include at least one colored indicator, which can easily be selected by a person skilled in the art.

In particular, said colored indicator can be selected from bromophenol blue, bromothymol blue, methylene blue, and methyl red.

According to one embodiment, the composition of the invention can further comprise a UV filter, preferably of the type used in cosmetics for absorbing UVA.

According to a variant embodiment, said composition can further comprise up to 5% of said UV filter.

In particular, said UV filter can be selected from ethoxylated ethyl-4-aminobenzoate, 3-benzylidene camphor, and mixtures thereof.

A second object of the present invention relates to a method for making a dental barrier, characterized in that it comprises the following stages:

a) application of a composition as defined previously on the dental tissues to be protected, in particular the gingival tissues, by means of an applicator nozzle,
b) irradiation with a photopolymerization lamp in the area of the dental tissues covered with said composition, until a color change of the composition is observed, indicating that polymerization has gone to completion.

The invention thus relates to the use of a composition according to the invention to obtain a dental barrier, which is generated in situ at the site of application of said composition.

A third object of the present invention relates to a dental barrier, characterized in that it is obtained according to the method as defined previously or starting from a precursor composition as defined previously.

The dental barriers obtained according to the invention display advantageous properties of adhesion of the dental barrier to the dental tissues on which it is applied, in particular the gingival tissues, providing optimal protection of the area of the mouth to be protected.

Moreover, the dental barriers obtained according to the invention are extremely easy to use, as they can easily be removed from the gums using tweezers at the end of the treatment.

A final object of the present invention relates to the use of a dental barrier as defined previously, characterized in that it makes it possible to protect the dental tissues, in particular the gums, notably during treatment in the dental office, in particular an office-bleaching or whitening treatment.

In particular, according to the invention, on the one hand, said barrier enables the dental tissues, in particular the gums, to be isolated electrically during treatment in the dental office, in particular an office-bleaching.

We may mention for example the electrophoretic whitening treatment described in patent FR 2 844 719. The barrier is then used as gingival protection, with the function of isolating the gums relative to the tooth to ensure that the current passes through the tooth.

In particular, according to the invention, on the other hand, said barrier enables the dental tissues, in particular the gums, to be isolated chemically, notably during treatment in the dental office, in particular an office-bleaching treatment.

In fact, a whitening treatment of the teeth generally uses high concentrations of hydrogen peroxide, which is a powerful oxidizing agent which can cause burns.

Moreover, in particular, according to the invention, said barrier enables the dental tissues, in particular the gums, to be isolated thermally, notably during treatment in the dental office, in particular an office-bleaching treatment.

In fact, a whitening treatment of the teeth generally uses a strong light source, which may generate considerable heat.

Moreover, according to the invention, said barrier enables the dental tissues, in particular the gums, to be isolated from exposure to UVA, which can be harmful during prolonged exposure, notably during treatment in the dental office, in particular an office-bleaching treatment.

Other aims, characteristics and advantages of the invention will become clear from the explanatory description given below, referring to the various examples of application of the invention, given purely for illustration and which therefore are not in any way intended to limit the scope of the invention. In the examples, all the percentages given are percentages by weight, the temperature is in degrees Celsius, the pressure is atmospheric pressure, unless stated otherwise.

### DESCRIPTION OF THE DRAWINGS

FIG. 1 shows the manner of application of the composition according to the invention (2) on the gums (1) by means of an applicator nozzle (3).

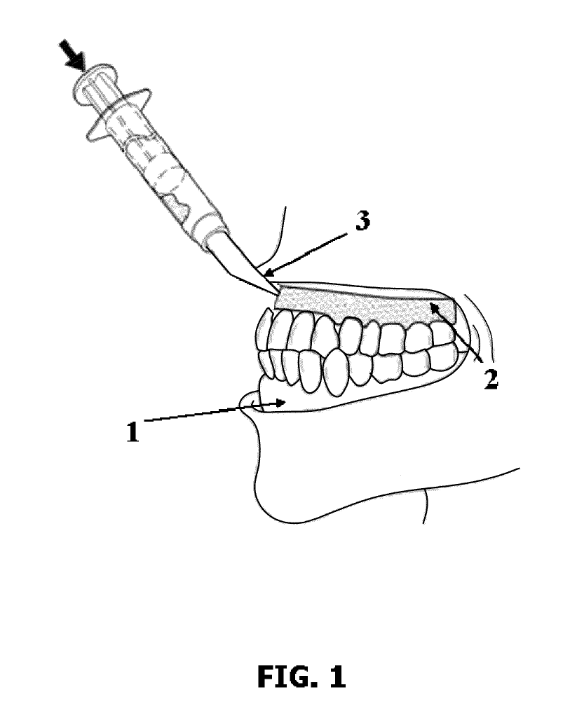

FIG. 2 shows, in chronological order, the polymerization of the dental dam (2′) from the composition (2), which is polymerized by irradiation with the photopolymerization lamp (4). A color change is observed between the polymerizable composition (2) and the polymerized composition (2′), due to the presence, in the composition, of a colored indicator which is monitoring the polymerization reaction.

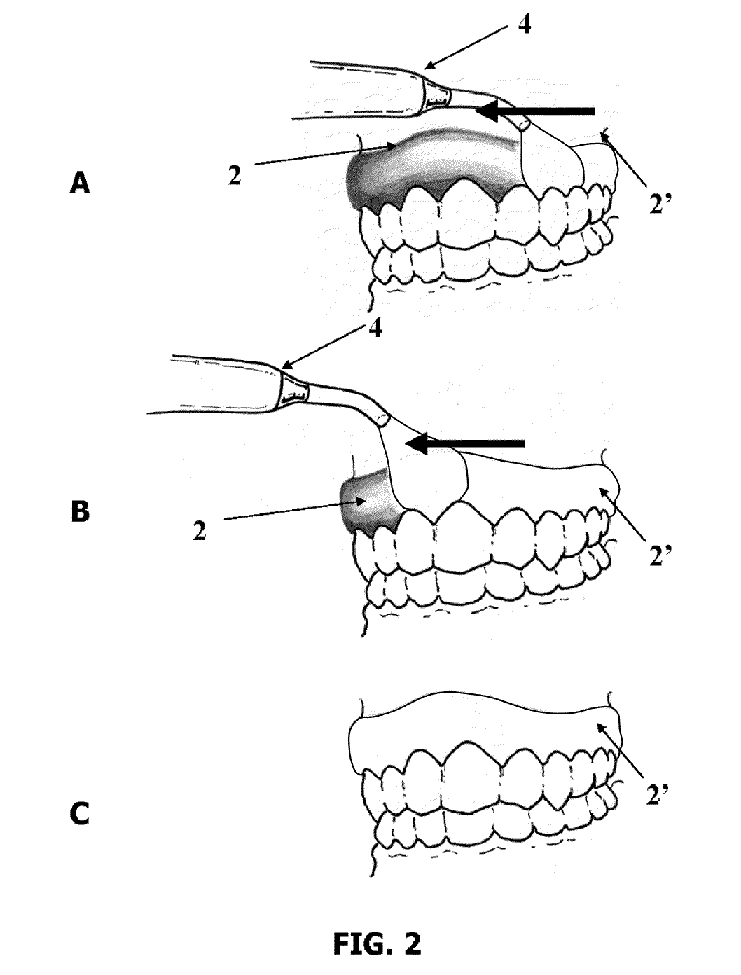

Drawings A and B show the progress of polymerization, by means of a photopolymerization lamp (4), of the composition according to the invention (2), which is gradually transformed into a dental barrier (2′).

Drawing C, which was made after complete polymerization of the composition (2), shows the dental barrier (2′) obtained, which covers the gums perfectly.

### EXAMPLES

### Equipment and Methods

### Equipment Used

The photopolymerization lamp is a lamp for dental use of the Mini LED SATELEC type, which has maximum power at a wavelength of 450 nm and a significant luminous intensity for wavelengths between 420 and 480 nm. The maximum optical power density of the lamp is 1100 mW/cm².

### Methods of Preparation of a Composition According to the Invention

The methods used for preparing the compositions according to the invention are methods that are well known by a person skilled in the art. Only a few particular examples will be mentioned here.

Synthesis of Composition a According to the Invention:

Mix together diurethane methacrylate, triethylene glycol dimethacrylate (monomer or monomers) and methylene blue (dye), then stir the mixture for 5 hours with a magnetic stirrer until the dye has dissolved completely. While stirring, slowly add the anhydrous colloidal silica (thickener) until the mixture is homogeneous. Finally, with protection from the light, add the ethyl-4-dimethylaminobenzoate and the camphorquinone (initiating system), then homogenize the mixture by stirring.

Synthesis of Composition E According to the Invention:

Mix together diurethane dimethacrylate, triethylene glycol dimethacrylate (monomer or monomers) and bromophenol blue (dye), then stir the mixture for 30 minutes with a magnetic stirrer until the dye has dissolved completely.
Then heat the mixture to 60° C. and switch on the stirrer, then gradually incorporate Compritol 888® (thickener) in the mixture until it melts and forms a homogeneous solution.
Then cool the mixture to room temperature.
Finally, while protected from the light, add the ethyl-4-dimethylaminobenzoate and the camphorquinone (initiating system), and homogenize the mixture by stirring.

### Method of Application of the Composition to Form a Dental Barrier According to the Invention

The composition according to the invention can be applied on the gums by means of an applicator nozzle, of the rubber-coating type. Then the practitioner can use a cannula to get the product between the teeth so that the gums to be protected are well covered. The practitioner can also use just a cannula directly for applying the composition.

Once the polymerizable composition has been applied, it is polymerized by means of a photopolymerization lamp, which is applied for some seconds above the composition to be polymerized. This irradiation permits activation of the polymerization reaction, and thus formation of the dental barrier. We also observe a color change between the polymerizable composition and the dental barrier obtained, due to the presence, in the composition, of an indicator that is colored according to the polymerization reaction.

### Examples of Compositions

### Example 1

### Composition A According to the Invention

A composition is prepared comprising:

| Product | wt. % |
|---|---|
| Diurethane dimethacrylate | 42.0% |
| Triethylene glycol dimethacrylate | 42.0% |
| Camphorquinone | 0.4% |
| Ethyl-4-dimethylaminobenzoate | 0.4% |
| Aerosil 200 ® | 15.1% |
| Methylene blue | 0.1% |

Here, diurethane dimethacrylate and triethylene glycol dimethacrylate are the monomers that constitute the copolymer.

The combination of camphorquinone and ethyl-4-dimethylaminobenzoate is able to initiate polymerization after exposure to a dental lamp emitting blue light of wavelength between 420 and 480 nm. This polymerization is responsible for adhesion to an organic substrate, for example a buccal tissue.

Ethyl-4-dimethylaminobenzoate is also able to absorb UV-A.

Aerosil 200®, which is an anhydrous colloidal silica, is used in the present invention in order to thicken the medium and promote its stability at rest, on account of its thixotropy.

Methylene blue acts as an indicator of polymerization, by its color change.

### Example 2

### Composition B According to the Invention

A composition is prepared comprising:

| Product | wt. % |
|---|---|
| Diurethane dimethacrylate | 59.0% |
| Triethylene glycol dimethacrylate | 25% |
| Camphorquinone | 0.4% |
| Ethyl-4-dimethylaminobenzoate | 0.4% |
| Aerosil ® | 15.1% |
| Bromophenol blue | 0.1% |

Same remarks as for composition A according to the invention.

Here, the bromophenol blue indicates polymerization by its color change.

### Example 3

### Composition C According to the Invention

A composition is prepared comprising:

| Product | wt. % |
|---|---|
| Bisphenol A dimethacrylate | 54.0% |
| Triethylene glycol dimethacrylate | 30.0% |
| Lucirin ® TPO | 0.8% |
| Ethoxylated ethyl-4-aminobenzoate | 5.0% |
| Aerosil 400 ® | 10.0% |
| Methyl red | 0.2% |

Here, bisphenol A dimethacrylate and triethylene glycol dimethacrylate are the monomers that constitute the copolymer.

Lucirin® TPO, which is 2,4,6-trimethylbenzoyldiphenylphosphine oxide, is able to initiate polymerization after exposure to a dental lamp emitting blue light of wavelength between 420 and 460 nm. This polymerization is responsible for the adhesion to an organic substrate, for example a buccal tissue.

Ethoxylated ethyl-4-aminobenzoate absorbs UV-A.

Aerosil 400®, which is a colloidal silica, is used in the present invention to thicken the medium.

Methyl red indicates polymerization by its color change.

### Example 4

### Composition D According to the Invention

A composition is prepared comprising:

| Product | wt. % |
|---|---|
| Methyl methacrylate | 40.0% |
| Urethane dimethacrylate | 44.0% |
| Bisacylphosphine oxide | 1.4% |
| 3-Benzylidene camphor | 1.5% |
| Aerosil 200 ® | 13.0% |
| Methylene blue | 0.1% |

Here, methyl methacrylate and urethane dimethacrylate are the monomers that constitute the copolymer.

Bisacylphosphine oxide is able to initiate polymerization after exposure to a dental lamp emitting blue light of wavelength between 400 and 440 nm. This polymerization is responsible for the adhesion to an organic substrate, for example a buccal tissue.

3-Benzylidene camphor absorbs UV-A.

Aerosil 200® in the present invention is used for thickening the medium.

Methylene blue indicates polymerization by its color change.

### Example 5

### Composition E According to the Invention

A composition is prepared comprising:

| Product | wt. % |
|---|---|
| Diurethane dimethacrylate | 40.0% |
| Triethylene glycol dimethacrylate | 39.6% |
| Camphorquinone | 0.4% |
| Ethyl-4-dimethylaminobenzoate | 0.4% |
| Compritol 888 ® | 19.1% |
| Bromophenol blue | 0.5% |

Same remarks as for examples 1 and 2.

Compritol 888®, which is a glycerol dibehenate, is used in the present invention to thicken the medium and promote its stability at rest.

1. A precursor composition for dental barrier, comprising at least one monomer of methacrylate or methacrylic derivative type, at least one thickener, a photochemical means of initiating polymerization, and at least one colored indicator for monitoring the polymerization reaction.

2. The composition of claim 1, comprising:
50 to 90 wt. % of at least one monomer,
10 to 49 wt. % of at least one thickener,
up to 2 wt. % of a photochemical means of initiating polymerization, and
up to 5 wt. % of a colored indicator.

3. The composition of claim 1, wherein said at least one monomer is a methacrylic monomer.

4. The composition of claim 3, wherein said monomer is selected from the group consisting of the alkylmethacrylate, the alkylhydroxymethacrylate and the alkylaminomethacrylate derivatives.

5. The composition of claim 3, wherein said monomer is selected from the group consisting of diurethane dimethacrylate, triethylene glycol dimethacrylate, bisphenol A dimethacrylate, methyl methacrylate, and mixtures thereof.

6. The composition of claim 3, wherein said monomer comprises a mixture constituted of diurethane dimethacrylate and triethylene glycol dimethacrylate.

7. The composition of claim 1, wherein said at least one thickener is selected from the group consisting of cetyl alcohol, anhydrous colloidal silica, glycerol dibehenate, alginic acid, sodium alginate, potassium alginate, ammonium alginate, calcium alginate, propane-1,2-diol alginate, agar, carrageenan, furcellaran, carob gum, oat gum, guar gum, tragacanth, gum arabic, xanthan gum, gum karaya, gellan gum, sorbitol, mannitol, glycerol, polyoxyethylene stearate, polyoxyethylene-20-sorbitan monolaurate, polyoxyethylene-20-sorbitan monooleate, polyoxyethylene-20-sorbitan monopalmitate, polyoxyethylene-20-sorbitan monostearate, polyoxyethylene-20-sorbitan tristearate, pectin, gelatin, cellulose, methyl cellulose, ethyl cellulose, hydroxypropyl cellulose, hydroxypropylmethyl cellulose, methylethyl cellulose, carboxymethyl cellulose, salts of fatty acids, mono- and diglyceride fatty acids, esters of mono- and diglycerides, sugars of esters of fatty acids, sugar glycerides, polyglycerol esters of fatty acids, polyglycerol polyricinoleate, propylene glycol esters of fatty acids, mixtures of glycerol and propylene glycol esters of lactic acid and of fatty acids, esterified soya oil, sorbitan stearate, sorbitan tristearate, sorbitan monolaurate, sorbitan monooleate, sorbitan monopalmitate and mixtures thereof.

8. The composition of claim 1, wherein said photochemical means of initiating polymerization is selected from the group consisting of camphorquinone, ethyl-4-dimethylaminobenzoate, bisacylphosphine oxide, 2,4,6-trimethylbenzoyldiphenylphosphine oxide and the camphorquinone/ethyl-4-dimethylaminobenzoate system.

9. The composition of claim 1, wherein said at least one colored indicator is selected from the group consisting of bromophenol blue, methylene blue, bromothymol blue and methyl red.

10. The composition of claim 1, wherein it further comprises a UV filter.

11. The composition of claim 10, wherein it further comprises up to 5% of said UV filter.

12. The composition of claim 10, wherein said UV filter is selected from the group consisting of ethoxylated ethyl-4-aminobenzoate, 3-benzylidene camphor and mixtures thereof.

13. A method for making a dental barrier, which comprises the following stages:
a) the application of the composition of claim 1 on the dental tissues to be protected, by means of an applicator nozzle,
b) the irradiation with a photopolymerization lamp on the area of the dental tissues covered with said composition, until a color change of the composition is observed, indicating that polymerization has gone to completion.

14. A dental barrier, which is obtained by the method of claim 13 or from the precursor composition of claim 1.

15. A method intended for protecting dental tissues during treatment in the dental office, wherein the dental barrier of claim 14 is applied on said dental tissues for protecting them.

16. The method of claim 15, wherein it is intended for isolating said dental tissues electrically, chemically, thermally, from UVA or from several types of isolations at the same time.

17. The method of claim 15, wherein said treatment is an office-bleaching treatment.
